(19) United States
(12) Patent Application Publication
(10) Pub. No.: US 2013/0220324 A1
(43) Pub. Date: Aug. 29, 2013

(54) SYSTEMS AND METHODS FOR PROVIDING OSCILLATORY PRESSURE CONTROL VENTILATION

(71) Applicants: Mehdi M. Jafari, Laguna Hills, CA (US); Rhomere S. Jimenez, Chula Vista, CA (US); Jeffrey K. Aviano, Escondido, CA (US); Edward R. McCoy, Vista, CA (US)

(72) Inventors: Mehdi M. Jafari, Laguna Hills, CA (US); Rhomere S. Jimenez, Chula Vista, CA (US); Jeffrey K. Aviano, Escondido, CA (US); Edward R. McCoy, Vista, CA (US)

(73) Assignee: Nellcor Puritan Bennett LLC, Boulder, CO (US)

(21) Appl. No.: 13/408,375

(22) Filed: Feb. 29, 2012

(51) Int. Cl. A61M 16/00

(52) U.S. Cl. 128/204.23; 128/204.18; 128/204.21

(57) ABSTRACT

A method for ventilating a patient with a ventilator includes receiving a target pressure input for a breathing phase, receiving at least one oscillation parameter, imposing an oscillatory waveform on the target pressure, the oscillatory waveform having characteristics defined by the at least one oscillation parameter and configured to oscillate substantially about the target pressure for at least a portion of the breathing phase, and delivering an amount of flow sufficient to achieve an oscillatory target pressure based on the imposed oscillatory waveform.

### INTRODUCTION

Medical ventilator systems have long been used to provide ventilatory and supplemental oxygen support to patients. These ventilators typically comprise a source of pressurized oxygen which is fluidly connected to the patient through a conduit or tubing. Traditional pressure control ventilation delivers an inspiration, an exhalation, or both with a fixed constant target pressure. In some instances, it would be beneficial to vary the target pressure to trigger increased alveolar recruitment and perfusion.

### Oscillatory Pressure Control Ventilation

This disclosure describes systems and methods for providing oscillatory pressure control during ventilation of a patient to optimize respiratory recruitment and perfusion.

In part, this disclosure describes a method for ventilating a patient with a ventilator. The method includes: receiving a target pressure input for a breathing phase, receiving at least one oscillation parameter, imposing an oscillatory waveform on the target pressure, the oscillatory waveform having characteristics defined by the at least one oscillation parameter and configured to oscillate substantially about the target pressure for at least a portion of the breathing phase, and delivering an amount of flow sufficient to achieve an oscillatory target pressure based on the imposed oscillatory waveform.

The disclosure further describes a ventilator system that includes: a pressure generating system adapted to generate a flow of breathing gas, a ventilation tubing system including a patient interface for connecting the pressure generating system to a patient, at least one sensor operatively coupled to at least one of the pressure generating system, the patient, and the ventilation tubing system, an oscillatory pressure control module configured to receive a target pressure input for a breathing phase, receive at least one oscillation parameter, detect that an amount of flow sufficient to achieve the target pressure has been achieved, impose an oscillatory waveform on the target pressure, the oscillatory waveform having characteristics defined by the at least one oscillation parameter and configured to oscillate substantially about the target pressure for at least a portion of a breathing phase, and deliver an amount of flow sufficient to achieve an oscillatory target pressure based on the imposed oscillatory waveform, and a processor in communication with the pressure generating system, the at least one sensor, and the oscillatory pressure control module.

The disclosure additionally describes a computer-readable medium having computer-executable instructions for performing a method for ventilating a patient with a ventilator. The method includes: receiving a target pressure input for a breathing phase, receiving at least one oscillation parameter, imposing an oscillatory waveform on the target pressure, the oscillatory waveform having characteristics defined by the at least one oscillation parameter and configured to oscillate substantially about the target pressure for at least a portion of the breathing phase, and delivering an amount of flow sufficient to achieve an oscillatory target pressure based on the imposed oscillatory waveform.

The disclosure additionally describes a further method for ventilating a patient with a ventilator. The method includes: receiving a target tidal volume input, receiving a first target pressure input, receiving at least one oscillation parameter, imposing a first oscillatory waveform on the target pressure, the first oscillatory waveform defined by the at least one oscillation parameter and configured to oscillate substantially about the first target pressure for at least a portion of a first breathing phase, delivering a first amount of flow sufficient to achieve a first oscillatory target pressure based on the first imposed oscillatory waveform, after a first flow delivery is substantially complete, estimating an amount of volume delivered, based on the estimated delivered volume, adjusting the first pressure input, imposing a second oscillatory waveform on the adjusted target pressure, the second oscillatory waveform configured to oscillate substantially about the adjusted target pressure for at least a portion of a second breathing phase, and delivering a second amount of flow sufficient to achieve an adjusted target pressure based on the second imposed oscillatory waveform.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of embodiments of systems and methods described below and are not meant to limit the scope of the invention in any manner, which scope shall be based on the claims.

FIG. 1 is a diagram illustrating an embodiment of an exemplary ventilator connected to a human patient.

FIG. 2 is a block-diagram illustrating an embodiment of a ventilatory system having a user interface for operating a ventilator in oscillatory pressure control mode.

FIG. 3 illustrates an embodiment of a graph of oscillatory pressure controlled delivered pressure over time during an inhalation.

FIG. 4 illustrates a further embodiment of a graph of oscillatory pressure controlled delivered pressure over time during an inhalation.

FIG. 5 is an illustrative flow for operating a ventilator in oscillatory pressure control mode.

FIGS. 6A-6B are illustrative flows for operating a ventilator in oscillatory pressure control mode.

### DETAILED DESCRIPTION

Although the techniques introduced above and discussed in detail below may be implemented for a variety of medical devices, the present disclosure will discuss the implementation of these techniques for use in a mechanical ventilator system. The reader will understand that the technology described in the context of a ventilator system could be adapted for use with other therapeutic equipment providing oscillatory pressure controlled delivery of gas flow to a patient.

This disclosure describes systems and methods for configuring a ventilator to determine oscillatory pressure controlled delivery of gas flow to a patient. According to embodiments, an average target pressure may be determined and an oscillating waveform may be imposed substantially about the average target pressure value. Upper and lower oscillatory bounds may be defined, as well as a time duration for a single oscillation. Oscillatory waveform may dynamically adapt to changes in patient parameters on a breath by breath basis to provide optimum gas flow to a patient.

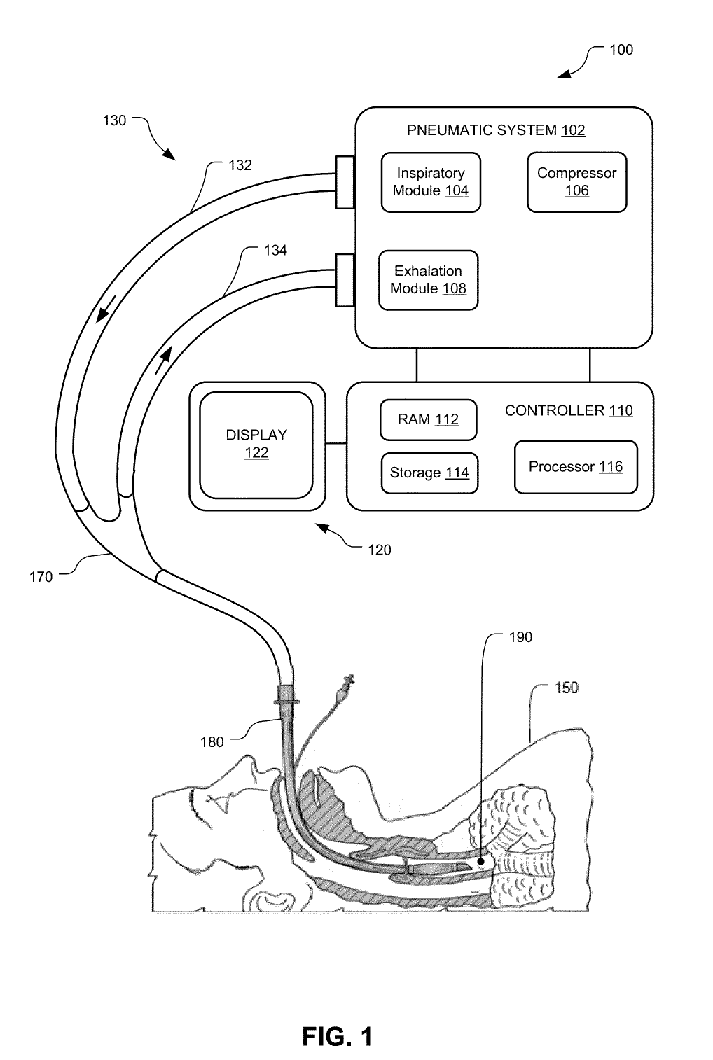

FIG. 1 is a diagram illustrating an embodiment of an exemplary ventilator 100 connected to a human patient 150.

Ventilator 100 includes a pneumatic system 102 (also referred to as a pressure generating system 102) for circulating breathing gases to and from patient 150 via the ventilation tubing system 130, which couples the patient to the pneumatic system via an invasive (e.g., endotracheal tube, as shown, or a tracheostomy tube) or a non-invasive (e.g., nasal mask) patient interface.

Ventilation tubing system 130 may be a two-limb (shown) or a one-limb circuit for carrying gases to and from the patient 150. In a two-limb embodiment, a fitting, typically referred to as a “wye-fitting” or “patient wye” 170, may be provided to couple an invasive patient interface 180 (as shown, an endotracheal tube) or a non-invasive (NIV) patient interface (e.g., mask, not shown) to an inspiratory limb 132 and an expiratory limb 134 of the ventilation tubing system 130.

Pneumatic system 102 may be configured in a variety of ways. In the present example, system 102 includes an exhalation module 108 coupled with the expiratory limb 134 and an inhalation module 104 coupled with the inspiratory limb 132. Compressor 106 or other source(s) of pressurized gases (e.g., air, oxygen, and/or helium) is coupled to inhalation module 104 to provide a gas source for ventilatory support via inspiratory limb 132.

The pneumatic system 102 may include a variety of other components, including mixing modules, valves, sensors, tubing, accumulators, filters, etc. Controller 110 is operatively coupled with pneumatic system 102, signal measurement and acquisition systems, and an operator interface 120 that may enable an operator to interact with the ventilator 100 (e.g., change ventilatory settings, select operational modes, view monitored parameters, etc.). Controller 110 may include memory 112, one or more processors 116, storage 114, and/or other components of the type commonly found in command and control computing devices. In the depicted example, operator interface 120 includes a display 122 that may be touch-sensitive and/or voice-activated, enabling the display 122 to serve both as an input and output device.

The memory 112 includes non-transitory, computer-readable storage media for storing software that is executed by the one or more processors 116 and which controls the operation of the ventilator 100. In an embodiment, the memory 112 includes one or more solid-state storage devices such as flash memory chips. In an alternative embodiment, the memory 112 may be mass storage connected to the one or more processors 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the one or more processors 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the ventilatory system or between the ventilatory system and other therapeutic equipment and/or remote monitoring systems may be conducted over a distributed network via wired or wireless means. Further, the present methods may be configured as a presentation layer built over the TCP/IP protocol. TCP/IP stands for “Transmission Control Protocol/Internet Protocol” and provides a basic communication language for many local networks (such as intra- or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination.

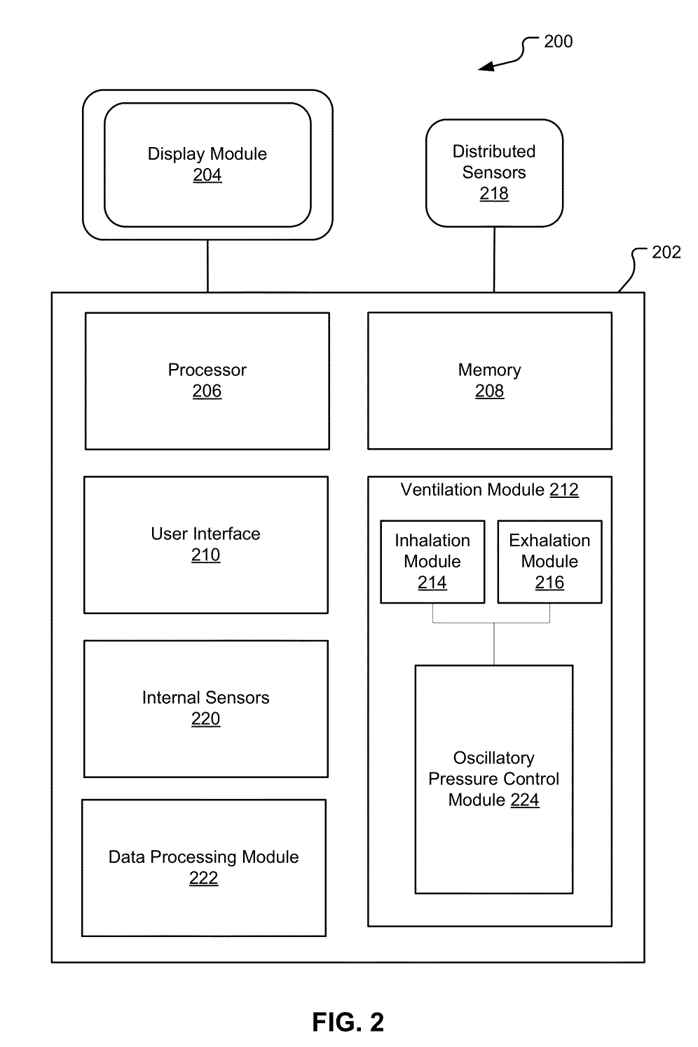

FIG. 2 is a block-diagram illustrating an embodiment of a ventilatory system for providing oscillatory pressure controlled delivery of gas flow to a patient.

Ventilatory system 200 includes ventilator 202 with its various modules and components. That is, ventilator 202 may further include, inter alia, one or more processors 206, memory 208, user interface 210, and ventilation module 212 (which may further include and/or communicate with inspiration module 214 and exhalation module 216). The one or more processors 206 are defined as described above for one or more processors 116. Processors 206 may further be configured with a clock whereby elapsed time may be monitored by the system 200. Memory 208 is defined as described above for memory 112.

The ventilatory system 200 may also include a display module 204 communicatively coupled to ventilator 202. Display module 204 may provide various input screens, for receiving clinician input, and various display screens, for presenting useful information to the clinician. The display module 204 is configured to communicate with user interface 210 and may include a graphical user interface (GUI). The GUI may be an interactive display, e.g., a touch-sensitive screen or otherwise, and may provide various windows (i.e., visual areas) comprising elements for receiving user input and interface command operations and for displaying ventilatory information (e.g., ventilatory data, alerts, patient information, parameter settings, etc.). The elements may include controls, graphics, charts, tool bars, input fields, etc. Alternatively, other suitable means of communication with the ventilator 202 may be provided, for instance by a wheel, keyboard, mouse, or other suitable interactive device. Thus, user interface 210 may accept commands and input through display module 204. Display module 204 may also provide useful information in the form of various ventilatory data regarding the physical condition of a patient and/or a prescribed respiratory treatment. The useful information may be derived by the ventilator 202, based on data collected by a data processing module 222, and the useful information may be displayed to the clinician on display module 204 in the form of graphs, wave representations, pie graphs, or other suitable forms of graphic display.

Ventilation module 212 may oversee ventilation of a patient according to ventilatory settings. Ventilatory settings may include any appropriate input for configuring the ventilator to deliver breathable gases to a particular patient. Ventilatory settings may be entered by a clinician, e.g., based on a prescribed treatment protocol for the particular patient, or automatically generated by the ventilator, e.g., based on attributes (i.e., age, diagnosis, ideal body weight, gender, etc.) of the particular patient according to any appropriate standard protocol or otherwise. For example, ventilatory settings may include, inter alia, inspiratory pressure (P_{I}), pressure support (P_{SUPP}), rise time percent (rise time %), positive end-expiratory pressure (PEEP), etc.

Ventilation module 212 may further include an inspiration module 214 configured to deliver gases to the patient according to prescribed ventilatory settings. Specifically, inspiration module 214 may correspond to or control the inhalation module 104 or may be otherwise coupled to source(s) of pressurized gases (e.g., air, oxygen, and/or helium), and may deliver gases to the patient. Inspiration module 214 may be configured to provide ventilation according to various ventilatory types and modes, e.g., via volume-targeted, pressure-targeted, or via any other suitable type of ventilation. According to some embodiments, inspiration module 214 may be configured to deliver mandatory ventilation to a patient based on a set inspiratory volume or pressure for a set period of time (referred to as the inspiratory time, T_{I}). Alternatively, inspiration module 214 may be configured to deliver spontaneous ventilation to a patient based on an inspiratory pressure support setting. An inspiratory pressure support setting may be a set percentage of ventilation support, a set value of pressure support, or other suitable partial to full ventilation setting. According to additional embodiments, various ventilator control algorithms may control inspiration module 214 to maintain a target pressure during inspiration. According to some embodiments, the target pressure may be determined or calculated to minimize the work of breathing due to the breathing tube.

Ventilation module 212 may further include an exhalation module 216 configured to release gases from the patient's lungs according to prescribed ventilatory settings. Specifically, exhalation module 216 may correspond to or control exhalation module 108 or may otherwise be associated with and/or control an exhalation valve for releasing gases from the patient. By way of general overview, a ventilator may initiate exhalation based on lapse of an inspiratory time setting (T_{I}) or other cycling criteria set by the clinician or derived from ventilatory settings. Alternatively, exhalation may be cycled based on detection of patient effort or otherwise. Upon initiating the exhalation phase, exhalation module 216 may allow the patient to exhale by controlling an exhalation valve.

According to some embodiments, the inspiration module 214 and/or the expiration module 216 may be configured to synchronize ventilation with a spontaneously-breathing, or triggering, patient. Specifically, the ventilator may detect patient effort via a pressure-monitoring method, a flow-monitoring method, direct or indirect measurement of nerve impulses, or any other suitable method. Sensing devices may be either internal or distributed and may include any suitable sensing device, as described further herein. In addition, the sensitivity of the ventilator to changes in pressure and/or flow may be adjusted such that the ventilator may properly detect the patient effort, i.e., the lower the pressure or flow change setting the more sensitive the ventilator may be to patient triggering.

According to embodiments, a pressure-triggering method may involve the ventilator monitoring the circuit pressure, as described above, and detecting a slight drop in circuit pressure. The slight drop in circuit pressure may indicate that the patient's respiratory muscles are creating a slight negative pressure gradient between the patient's lungs and the airway opening in an effort to inspire. The ventilator may interpret the slight drop in circuit pressure as patient effort and may consequently initiate inspiration by delivering respiratory gases.

Alternatively, the ventilator may detect a flow-triggered event. Specifically, the ventilator may monitor the circuit flow, as described above. If the ventilator detects a slight drop in flow during exhalation, this may indicate, again, that the patient is attempting to inspire. In this case, the ventilator is detecting a drop in bias flow (or baseline flow) attributable to a slight redirection of gases into the patient's lungs (in response to a slightly negative pressure gradient as discussed above). Bias flow refers to a constant flow existing in the circuit during exhalation that enables the ventilator to detect expiratory flow changes and patient triggering. For example, while gases are generally flowing out of the patient's lungs during expiration, a drop in flow may occur as some gas is redirected and flows into the lungs in response to the slightly negative pressure gradient between the patient's lungs and the body's surface. Thus, when the ventilator detects a slight drop in flow below the bias flow by a predetermined threshold amount (e.g., 2 L/min below bias flow), it may interpret the drop as a patient trigger and may consequently initiate inspiration by delivering respiratory gases.

The ventilatory system 200 may also include one or more distributed sensors 218 communicatively coupled to ventilator 202. Distributed sensors 218 may communicate with various components of ventilator 202, e.g., ventilation module 212, internal sensors 220, data processing module 222, oscillatory pressure control module 224, and any other suitable components and/or modules. Distributed sensors 218 may be placed in any suitable location, e.g., within the ventilatory circuitry or other devices communicatively coupled to the ventilator. For example, sensors may be affixed to the ventilatory tubing or may be embedded in the tubing itself. Additionally or alternatively, sensors may be affixed or embedded in or near patient wye 170 and/or patient interface 180, as described above. A sensor affixed near the patient wye 170 may be configured to measure an actual wye pressure. Distributed sensors 218 may include pressure transducers for detecting circuit pressure, flowmeters for detecting circuit flow, optical or ultrasound sensors for measuring gas characteristics or other parameters, or any other suitable sensory device.

Ventilator 202 may further include one or more internal sensors 220. Similar to distributed sensors 218, internal sensors 220 may communicate with various components of ventilator 202, e.g., ventilation module 212, data processing module 222, oscillatory pressure control module 224, and any other suitable components and/or modules. Internal sensors 220 may employ any suitable sensory or derivative technique for monitoring one or more parameters associated with the ventilation of a patient. However, as opposed to the distributed sensors 218, the internal sensors 220 may be placed in any suitable internal location, such as, within the ventilatory circuitry or within components or modules of ventilator 202. For example, sensors may be coupled to the inhalation and/or exhalation modules, the exhalation valve, etc., for detecting pressure and/or flow. Specifically, internal sensors may include pressure transducers and flowmeters for measuring changes in pressure and airflow. Additionally or alternatively, internal sensors may utilize optical or ultrasound techniques for measuring changes in ventilatory parameters.

Ventilator 202 may further include a data processing module 222. As noted above, distributed sensors 218 and/or internal sensors 220 may collect data regarding various ventilatory parameters. A ventilatory parameter refers to any factor, characteristic, or measurement associated with the ventilation of a patient, whether monitored by the ventilator or by any other device. According to embodiments, internal and/or distributed sensors may further transmit collected data to the data processing module 222 and the data processing module 222 may be configured to measure data regarding some ventilatory parameters, to retrieve data regarding some ventilatory parameters or settings, to calculate data regarding other ventilatory parameters, and/or to graphically represent measured, retrieved, and/or calculated data on display module 204. According to embodiments, any measured, retrieved, calculated, and/or graphically represented data may be referred to as ventilatory data.

For example, according to some embodiments, the ventilator may periodically or continuously measure ventilatory data associated with pressure and/or flow in the patient circuit or in the breathing tube. According to additional embodiments, the ventilator may retrieve ventilatory data associated with ventilatory settings (e.g., O₂%, PEEP, P_{I}, P_{SUPP}, etc.), patient data (e.g., ideal body weight, IBW), breathing tube data (e.g., diameter, length, type, and/or resistance of breathing tube), atmospheric pressure data (e.g., measured or default of 0 cmH₂O), humidity data (e.g., measured or default of 100% saturation), etc. Retrieved ventilatory data may be acquired from any suitable database or data storage location associated with the ventilator (e.g., stored in memory 208, stored on a server accessible over a network, etc.). According to additional embodiments, the ventilator may periodically or continuously calculate ventilatory data, e.g., a pressure drop (ΔP) across the breathing tube (e.g., from the patient wye to the carina), a pressure at the patient wye a resistance associated with the breathing tube (e.g., based on length, size, type of breathing tube), a gas density (e.g., based on O₂%), etc.

As should be appreciated, the various modules described above do not represent an exclusive array of modules. Indeed, any number of additional modules may be suitably configured to execute one or more of the above-described operations within the spirit of the present disclosure. Furthermore, the various modules described above do not represent a necessary array of modules. Indeed, any number of the disclosed modules may be appropriately replaced by other suitable modules without departing from the spirit of the present disclosure. According to some embodiments, operations executed by the various modules described above may be stored as computer-executable instructions in the ventilator memory, e.g., memory 112, which computer-executable instructions may be executed by one or more processors, e.g., processors 116, of the ventilator.

Ventilation module 212 may further include an oscillatory pressure control module 224. According to some embodiments, oscillatory pressure control module 224 may be configured to generate oscillations during an inhalation period, an exhalation period, or both. Delivered oscillatory pressure may provide increased alveolar recruitment and perfusion. Oscillatory pressure control module 224 may receive one or more inputs relating to one or more patient parameters. In one embodiment, the oscillatory pressure control module 224 may receive a target pressure input defining a target pressure (hereinafter sometimes referred to as oscillatory target pressure or Pₘₑₐₙ). For instance, an operator (e.g., a clinician) of ventilation system 200 may input a target pressure value into ventilatory system 200 via user interface 210, and the target pressure value may be transferred to oscillatory pressure control module 224. Target pressure value may correspond to an average amount of pressure desired to be delivered to the patient (e.g., in which case the target flow may be determined by the ventilator from the target pressure). The desired target pressure may be set by the clinician (Pressure Control) or adaptively derived from a target tidal volume entered by a clinician (Volume-Targeted Pressure-Controlled). In some instances, target pressure is an average or mean of a desired delivered pressure. Target pressure may be an inspiratory target pressure or an expiratory target pressure (e.g., during exhalation, the target pressure may be decreased to a pre-selected positive end expiratory pressure (PEEP) level, depending on the desires of the operator). Target pressure may be determined based on one or more patient parameters, may be determined by an operator, and/or may be adaptively modified on a breath-by-breath basis to optimize a qualitative outcome for the patient.

Oscillatory pressure control module 224 may further receive a breathing phase total time duration input (e.g., a total inspiratory time input and/or a total expiratory time input for a single delivered breath). For instance, an operator may input a breathing phase total time duration value into ventilatory system 200 via user interface 210, and the breathing phase total time duration value may be transferred to oscillatory pressure control module 224. Breathing phase total time may be determined based on one or more patient parameters, may be determined by an operator, or the patient respiratory behavior, and/or may be adaptively modified on a breath-by-breath basis to optimize a qualitative outcome for the patient. In some embodiments, inspiratory time and/or expiratory time may be patient determined. In such embodiments, oscillatory pressure control module 224 may be configured to estimate an inspiration end or an exhalation end and adapt the start or end of delivered oscillatory pressure accordingly.

Oscillatory pressure control module 224 may also be configured to receive one or more oscillation parameters. Oscillation parameters may define one or more characteristics of a waveform imposed on at least a portion of a breathing phase. For instance, one or more oscillation parameters may define upper and lower bounds of an oscillation imposed on a target inspiratory or expiratory pressure, as described above. In some embodiments, an operator may input one or more oscillation parameter values into ventilatory system 200 via user interface 210, and the one or more oscillation parameter values may be transferred to oscillatory pressure control module 224. Oscillation parameters may be fixed or may be stochastically (e.g., randomly) determined and applied to a breathing phase. Oscillation parameters may also adapt to changes in patient behavior or breathing. Defined and bounded oscillations may be imposed during inhalation only, exhalation only, or during both inhalation and exhalation. Parameters of the oscillatory waveform may be substantially similar during inhalation and/or exhalation, or may vary from inhalation to exhalation, from breath to breath, from a first set of breaths to a second set of breaths, or in any other desired pattern.

In some embodiments, oscillation parameters may include a maximum pressure parameter. To this end, oscillatory pressure control module 224 may be configured to receive a maximum pressure input (Pₘₐₓ). Maximum pressure input may be a maximum inspiratory pressure input, or a maximum expiratory pressure input. Maximum pressure input may be utilized to calculate a maximum oscillation amplitude (Pₐₘₚₗ) for an oscillation imposed on the target pressure input. For instance, the formed oscillatory waveform may include upper and lower bounds defined by the difference between the target pressure and the maximum inspiratory pressure (e.g., Pₐₘₚₗ=Pₘₐₓ−Pₘₑₐₙ). For instance, the amount of average pressure delivered during one cycle (e.g., one full oscillatory pressure increase and one full oscillatory pressure decrease about the mean target level) may be about the amount of pressure desired to be delivered based on the input target pressure. In some embodiments, maximum pressure may be constant over a set period of time, may vary from breath to breath, or may vary in any desired pattern. In some embodiments, maximum oscillation amplitude Pₐₘₚₗ be a percentage (e.g., 2%-25%) of the average pressure target level Pₘₑₐₙ.

In some embodiments, oscillation parameters may include an oscillatory waveform shape parameter. To this end, oscillatory pressure control module 224 may also receive an oscillatory waveform shape selection. Oscillatory waveform may be periodic or non-periodic. In an exemplary embodiment, oscillatory waveform may be a sinusoidal. Additional non limiting examples of oscillatory waveforms that may be generated and imposed on a target pressure include square, rectangular, triangular, sawtooth, pulse, trigger (e.g., a very narrow pulse), composite, and the like. One or more algorithms utilized to generate and impose a desired wave shape may be stored, for example, in memory 208 and retrieved when configuring ventilation module 212 to provide oscillatory pressure control via oscillatory pressure control module 224.

In some embodiments, oscillation parameters may include an oscillation cycle duration parameter. To this end, oscillatory pressure control module 224 may also be configured to receive an oscillation cycle duration input. Oscillation cycle duration input may configure the time duration of a single oscillation cycle (e.g., one pressure increase and one pressure decrease). Oscillation cycle duration may be constant over a period of time, may vary from inhalation or exhalation or breath to breath, or may vary in any desired pattern. In some embodiments, a range of 1-10 milliseconds for an oscillation cycle period may be suitable. Oscillatory pressure control module 224 may also receive an inspiratory or expiratory total time duration input. Total time duration input may be per inhalation, exhalation, or breath. Oscillatory pressure control module 224 may then determine a number of oscillations to generate per inhalation or exhalation based on the oscillation cycle duration input and the total time duration input.

It is further contemplated that additional oscillatory or ventilation parameters may be determined and input into ventilation module 212 prior to ventilation of a patient. Additionally, other ventilation parameters (e.g., rise time, breath rate, oxygen concentration, etc.) may be input at ventilation setup and may also adapt to changes in patient breathing. The above described parameters are in no way limiting to the scope of applicable parameters suitable for selection and use with the described systems and methods.

Upon receipt of the one or more oscillation parameters by the oscillatory pressure control module 224, at least one component of ventilation module 212 (e.g., inhalation module 214 or exhalation module 216) may receive an indication that setup is complete and gas flow is to be delivered. For instance, when in an inspiratory phase, the ventilator may raise the pressure in the ventilator tubing system so that an input target inspiratory pressure is applied. The duration of the inspiratory and expiratory phases may be determined based on patient effort or based on a preselected inspiratory time and breath rate. The at least one component of ventilation module 212 may then deliver an amount of gas to reach the target pressure. Upon delivery, oscillatory pressure control module 224 may detect that the target pressure has been achieved. Oscillatory pressure control module 224 may then generate and impose an oscillating waveform on the value input target pressure for at least a portion of the received or detected time duration for the single inhalation or exhalation. For instance, the input target pressure value may be utilized in any suitable algorithm configured to generate an oscillatory target pressure based on the one or more input oscillation parameters. Determined oscillatory target pressure values may then be utilized by oscillatory pressure control module 224 to modulate the input target pressure according to the algorithm. In embodiments where the waveform is sinusoidal, and oscillatory pressure is provided for an inspiration, target pressure may be defined by the following equation:

Target Pressure=Pₘₑₐₙ+Pₐₘₚₗ*Sin(2pi*tᵢ*F)

Where Pₘₑₐₙ=target pressure, Pₐₘₚₗ=Pₘₐₓ−Pₘₑₐₙ, pi=3.1415926, tᵢ=inspiratory time, F=sinusoid frequency=1/T, and T=sinusoid period.

In some embodiments, oscillatory pressure control module 224 may be configured to increase the input target pressure up to the maximum pressure input value for half the waveform duration and subsequently decrease pressure an amount substantially equivalent to the pressure increase for the remaining time of the waveform duration. Thus, the delivered pressure may oscillate (e.g., incrementally increase or decrease) substantially about the input target pressure during a single inhalation, a single exhalation, or a single breath according to the one or more pre-determined or adaptive oscillation parameters described above. When the input time duration has expired, or when an inspiration or exhalation end is detected, oscillatory pressure control module 224 may cease delivery of oscillatory pressure until a next time period for oscillatory pressure delivery is detected.

Oscillatory pressure control module 224 may also be configured to provide oscillatory pressure during pressure regulated volume targeted ventilation. Generally, pressure regulated volume targeted ventilation is utilized to deliver an accurate set tidal volume to a patient in a respiratory ventilation system (e.g., ventilation system 200). The ventilation system may determine a pressure level corresponding with the desired delivered volume into the lungs. Within a breath, pressure may be regulated, but across breaths the target pressure may be modified so that a set volume enters the lungs. A target pressure delivered during a breathing phase (e.g., an inhalation or exhalation) may be determined based on the desired lung volume to be delivered.

During setup, an operator may select a volume of gas to be delivered to the patient, (e.g., delivered into the lung), over a specified time period such as every single inhalation, a minute, a number of breaths, etc. In some instances, a specific respiratory volume value (e.g., a tidal volume) may be input via user interface 210. This volume value is received by the ventilator and may be stored in memory 208 for use during ventilation. Ventilation module 212 may determine a target pressure level that will deliver the desired volume into the lungs. During a breathing phase, the target pressure may be set to an initial target pressure value via inhalation module 214. For instance, inhalation module 214 may receive an inhalation pressure desired value. This inhalation pressure desired value may be stored in memory 208. The desired value for the exhalation pressure may be received by exhalation module 216 and may also be stored in memory 208.

Target pressure may be an oscillatory target pressure, as discussed above. Oscillatory target pressure may be configured as described above. To this end, input breathing phase time, tidal volume amount, target pressure and any other parameters may be received by, for example, oscillatory pressure control module 224. Other parameters, including total breath time, rise time, oxygen concentration, and breath rate may be determined at setup. Any of the ventilation parameters (e.g., target pressure, maximum pressure, breath duration, etc.) may be constant (e.g., subject to a set desired volume) or may adaptively adjust to comply with one or more parameters (e.g., satisfy delivery of a certain volume while oscillating about the target). One or more input parameters may also adapt based on changes in patient behavior (e.g., spontaneous breathing). Delivered volume may remain constant or substantially constant while the target pressure per breathing phase for each breath oscillates according to a previously determined setting.

To provide pressure regulated volume targeted ventilation with oscillatory pressure control, an initial amount of pressure may be delivered to the patient during an inspiration or exhalation phase (via, for example, ventilation module 212). Within a single breathing phase, delivered pressure may oscillate substantially about the target pressure level, which may be constant for the duration of the breathing phase. After delivery of a breath, a delivered volume may be estimated. A delivered volume may be measured, for instance, by one or more distributed sensors 218, or one or more internal sensors 220. The determined delivered volume may be compared to the target volume value to determine if the delivered volume is less than or greater than the target volume. Breath to breath, the oscillatory target pressure may be adjusted to reach the target volume. For instance, based on the estimated delivered volume information, during a next breath the oscillatory target pressure may be varied to increase or decrease delivered pressure so that a set volume enters the lungs.

The oscillatory target pressure (Pₘₑₐₙ) may be increased or decreased by a preselected pressure increment or another algorithm such as any appropriate form of closed-loop dynamic regulation during the next breathing phase. The oscillatory target pressure value may be increased or decreased until the delivered volume is substantially equal to the volume target (e.g., to maintain a constant volume of delivered gas that enters the patient's lungs). Increasing the oscillatory target pressure may provide an indication that more volume is to be delivered. Decreasing the oscillatory target pressure may provide an indication that less volume is to be delivered. The delivered oscillatory pressure may also be adjusted adaptively so that after several breaths, the volume delivered to the patient has reached a level equivalent or substantially equivalent to the set volume. Oscillations may similarly adjust (e.g., increase or decrease in amplitude) according to the target pressure for each breath based on the set volume target desired across multiple breaths.

To provide oscillatory pressure regulated volume targeted ventilation, during a next breath, an amount of flow sufficient to achieve the adjusted target pressure (e.g., an amount of flow greater than or less than the amount of flow delivered in a previous breath) may be delivered. For instance, an oscillatory waveform (having similar or different parameters as oscillatory waveform imposed on the first target pressure) may be imposed on the adjusted target pressure via, for instance, oscillatory pressure control module 224. The oscillatory waveform may include upper and lower bounds defined by, for example, a second maximum pressure input, or any other adjusted oscillatory parameters (if adjusted) as described above. At least one component of ventilation module 212 may adjust the target pressure such that an adjusted oscillatory target pressure amount is delivered. The oscillatory waveform may then be configured to oscillate substantially about the adjusted target pressure.

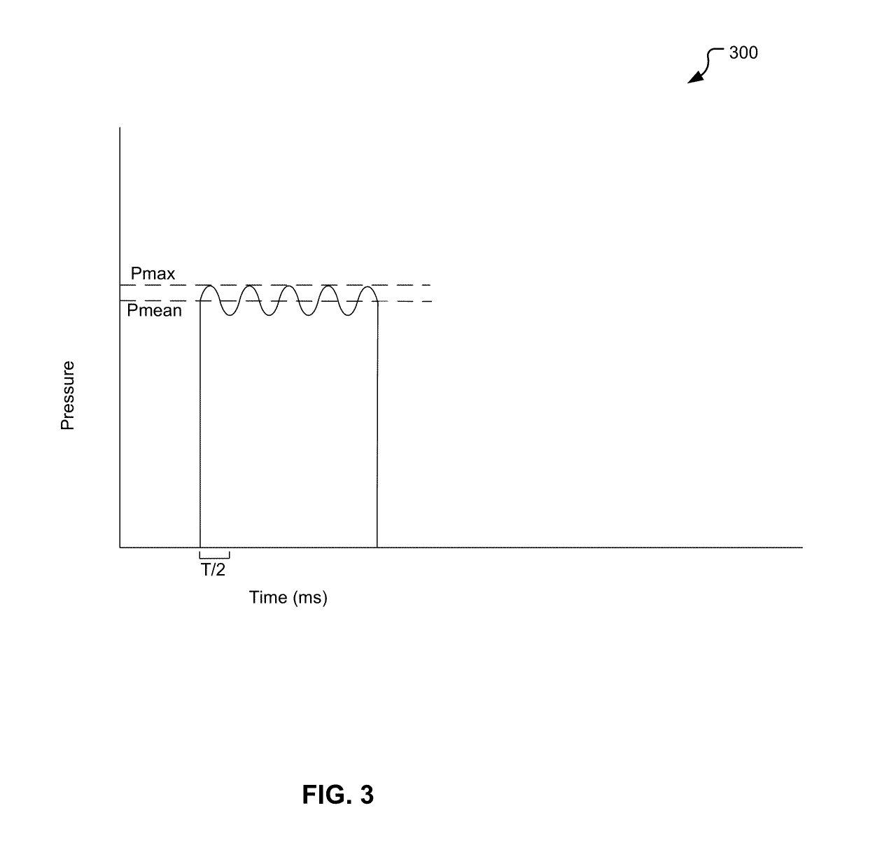

FIG. 3 illustrates an embodiment of a graph of oscillatory pressure controlled delivered pressure over time during an inhalation. The embodiment shown in FIG. 3 is a non-limiting example of the above described system and method. In an embodiment as illustrated in FIG. 3, a target inspiration input value Pₘₑₐₙ may be set. Pressure may reach the target inspiration input value Pₘₑₐₙ during the first portion of an inhalation. After reaching the target inspiration input value Pₘₑₐₙ, as illustrated in FIG. 3, the inspiration pressure may be varied according to one or more set parameters to provide oscillatory delivery of gas pressure/flow. Variation in inspiration pressure may not exceed the difference between the target inspiration input value Pₘₑₐₙ and the maximum target inspiratory pressure Pₘₐₓ. The inspiratory pressure may then be decreased to signify the end of an inspiration or when an inspiratory end is detected.

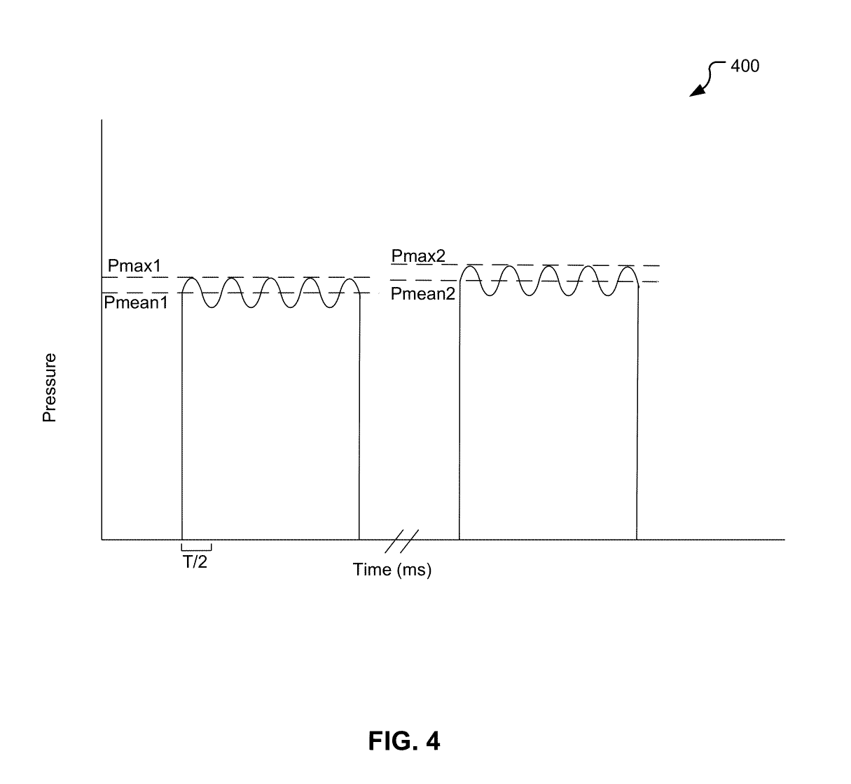

FIG. 4 illustrates a further embodiment of a graph of oscillatory pressure controlled delivered pressure over time in volume targeted ventilation. Specifically, FIG. 4 illustrates two subsequent inspiratory phases (exhalation not shown). The embodiment shown in FIG. 4 is a non-limiting example of the above described system and method. An amount of inspiratory tidal volume may be desired to be delivered to a patient. An inspiratory tidal volume input may be set. A first target inspiration input value Pₘₑₐₙ₁ may be set. Pressure may reach the first target inspiration input value Pₘₑₐₙ₁ during the first inhalation. After completing the first inspiration, as illustrated in FIG. 4, the inspiration pressure target for the next inspiration may be varied according to one or more set parameters to provide oscillatory delivery of gas flow. Variation in inspiration pressure may not exceed the difference between the first target inspiration input value Pₘₑₐₙ₁ and the first maximum target inspiratory pressure Pₘₐₓ₁. The inspiratory pressure may then be terminated at the end of an inspiration. An estimated amount of delivered volume may be determined. During a second breath, a second target inspiration input value Pₘₑₐₙ₂ may be set. Pressure may reach the second target inspiration input value Pₘₑₐₙ₂ during the inhalation. After completing the second target inspiration input value Pₘₑₐₙ₂, as illustrated in FIG. 4, the inspiration pressure may be varied according to one or more set parameters to provide oscillatory delivery of gas flow for the next breath. Variation in inspiration pressure may not exceed a second maximum target inspiratory pressure Pₘₐₓ₂. The inspiratory pressure may then be terminated at the end of an inspiration (e.g., when ventilation module 212 detects that a patient is beginning an exhalation). This process of increasing or decreasing the desired target pressure is continued until inspiratory tidal volume converges within an acceptable range of the desired tidal volume.

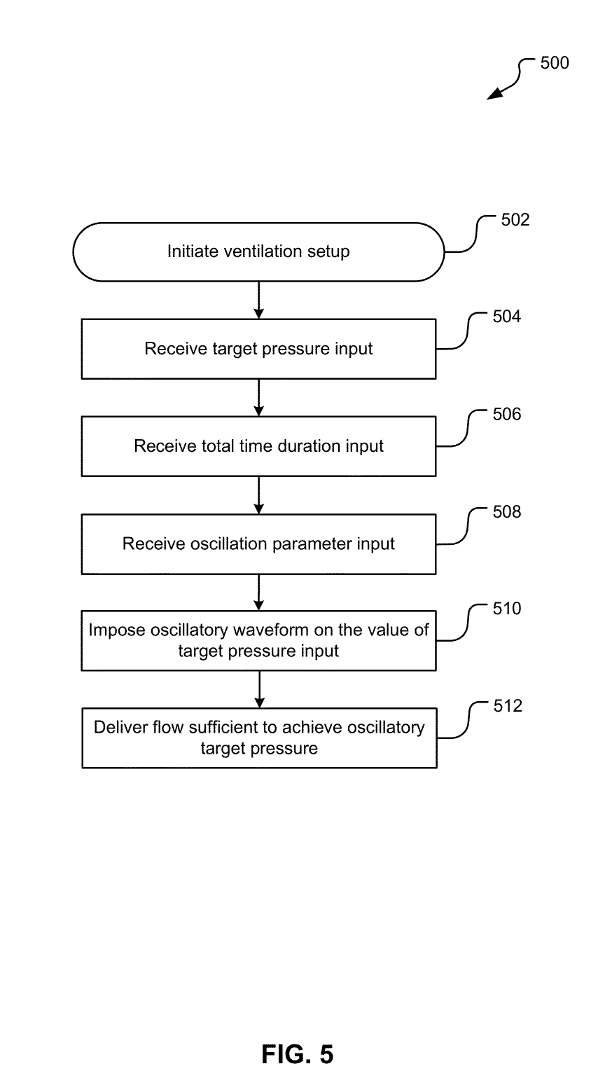

FIG. 5 is a flow chart illustrating an embodiment of a method 500 for providing oscillatory pressure controlled delivery of gas flow to a patient.

Method 500 begins with ventilation setup operation 502. According to embodiments, ventilation involves delivering breathing gases to a patient who is unable to breathe completely independently. Ventilation includes delivering any portion of breathing gases, from full mandatory ventilation to full or partially-supported spontaneous ventilation.

At receive operation 504, a target pressure input is received. For instance, as described above, oscillatory pressure control module 224 may receive a target pressure input. Target pressure value may be used to calculate an amount of flow desired to be delivered to the patient. Target pressure may be an inspiratory target pressure or an expiratory target pressure (e.g., during exhalation, the pressure may be dropped to some pre-selected positive end expiratory pressure (PEEP) level, depending on the desires of the operator). Target pressure may be determined based on one or more patient parameters and/or may be determined by a user.

At receive operation 506, a total time duration input may be received. As described above, oscillatory pressure control module 224 may receive a total inspiratory time input and/or a total expiratory time input for a single delivered breath. In some embodiments, inspiratory time and/or expiratory time may be patient determined. In such embodiments, oscillatory pressure control module 224 may be configured to determine an inspiration end or exhalation end and adapt the start or end of delivered oscillatory pressure accordingly.

At receive operation 508 one or more oscillation parameter inputs may be received. Oscillation parameters may be input into ventilating system 200, for example, via user interface 210 of FIG. 2. Oscillation parameters may define one or more characteristics of an imposed waveform. Oscillation parameters may be fixed or may be stochastically (e.g., randomly) determined. Oscillation parameters may further define upper and lower bounds of an imposed oscillation. Defined and bounded oscillations may be imposed during inhalation only, exhalation only, or during both inhalation and exhalation. Parameters of the oscillatory waveform (e.g., sine wave) may be substantially similar during inhalation and/or exhalation, or may vary from inhalation to exhalation, from breath to breath, from a first set of breaths to a second set of breaths, and the like. Oscillation parameters may be received oscillatory pressure control module 224. In some embodiments, received oscillation parameters by may include a maximum pressure parameter, a cycle duration or frequency parameter, and an oscillatory waveform shape parameter as described above. In some instances, waveform shape may be a sine wave. Additional non-limiting examples of oscillatory waveform shapes that may be generated and imposed on a target pressure include square, rectangular, triangular, sawtooth, pulse, trigger (??), composite, and the like. One or more algorithms utilized to generate and impose a desired waveform shape may be stored, for example, in memory 208 and retrieved when configuring ventilation module 212 to provide oscillatory pressure control via oscillatory pressure control module 224.

At impose operation 510, an oscillatory waveform may be imposed on the target pressure. Oscillatory pressure control module 224 may detect that the target pressure has been achieved. Alternatively, one or more distributed sensors 218 or internal sensors 220 may detect that the target pressure has been reached and transmit an indication to oscillatory pressure control module 224 for further processing. Oscillatory pressure control module 224 may then generate and impose an oscillating waveform. The oscillating waveform may include upper and lower bounds defined by the oscillation parameters. Individual oscillations of the oscillatory waveform may also be further defined by one or more oscillation parameters as described above. To this end the oscillating waveform may be configured to oscillate substantially about the target pressure for a set duration, including an inhalation, a plurality of consecutive inhalations, a single exhalation, a plurality of consecutive exhalations, a single breath or a plurality of consecutive breaths, or any other desired pattern of inhalation and/or exhalation. In some embodiments, oscillatory pressure control module 224 may impose the oscillating waveform on the value input target pressure for at least a portion of the received or detected time duration for the single inhalation or exhalation. For instance, the input target pressure value may be utilized in any suitable algorithm configured to generate an oscillatory pressure based on the one or more input oscillation parameters. For instance, in embodiments where the waveform is sinusoidal, and oscillatory pressure is provided for an inspiration, target pressure may be defined by the following equation:

Target Pressure=Pₘₑₐₙ+Pₐₘₚₗ*sin(2pi*tᵢ*F)

Where Pₘₑₐₙ=target pressure, Pₐₘₚₗ=Pₘₐₓ−Pₘₑₐₙ, pi=3.1415926, tᵢ=inspiratory time, F=sinusoid frequency-1/T, and T=sinusoid period.

Oscillatory target pressure values may then be utilized by oscillatory pressure control module 224 to modulate the input target pressure according to the algorithm. In some embodiments, oscillatory pressure control module 224 may be configured to increase the input target pressure up to the maximum pressure input value for half the waveform cycle duration and decrease inspiratory pressure an amount substantially equivalent to the pressure increase for the remaining time of the inspiratory waveform duration. Thus, the delivered pressure may oscillate (e.g., incrementally increase or decrease) substantially about the input target pressure during a single inhalation, a single exhalation, or a single breath according to the one or more pre-determined or adaptive oscillation parameters described above. When the input time duration has expired, or when an inspiration or exhalation end is detected, oscillatory pressure control module 224 may cease delivery of oscillatory pressure until a next time period for delivery is detected.

At deliver operation 512, a gas flow may be delivered to a patient sufficient to reach the oscillatory target pressure. In some embodiments, at least one component of ventilation module 212 (e.g., inhalation module 214 or exhalation module 216) may receive an indication that gas flow is to be delivered. For instance, when in an inspiratory phase, the ventilator raises the pressure in the ventilator tubing system so that the input target inspiratory pressure is applied. The at least one component of ventilation module 212 may then deliver an amount of gas to reach the target pressure.

As should be appreciated, the particular steps and methods described above with reference to FIG. 5 are not exclusive and, as will be understood by those skilled in the art, the particular ordering of steps as described herein is not intended to limit the method, e.g., steps may be performed in differing order, additional steps may be performed, and disclosed steps may be excluded without departing from the spirit of the present methods.

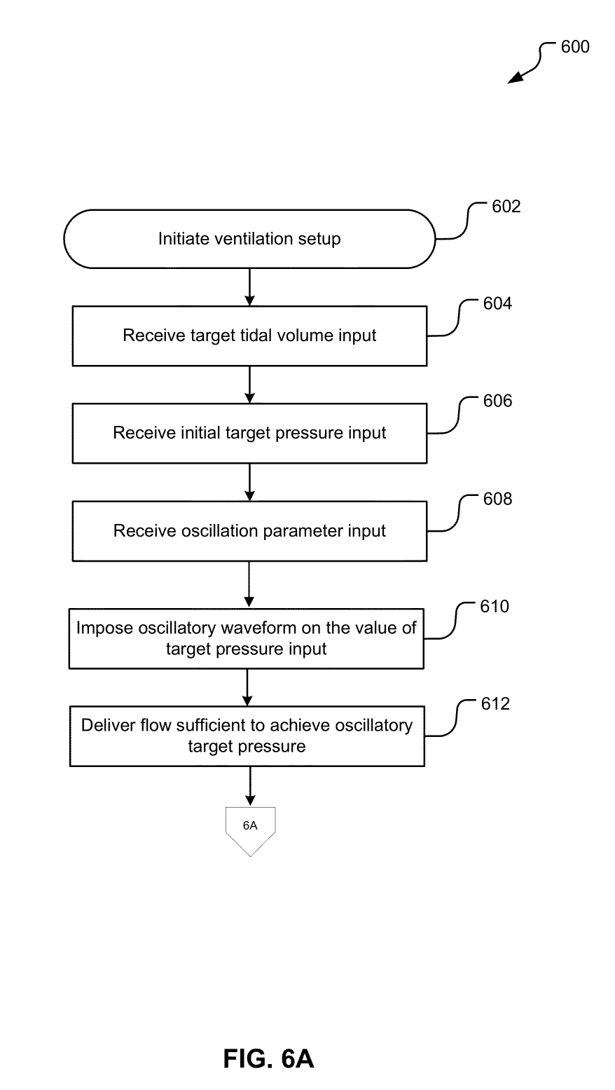

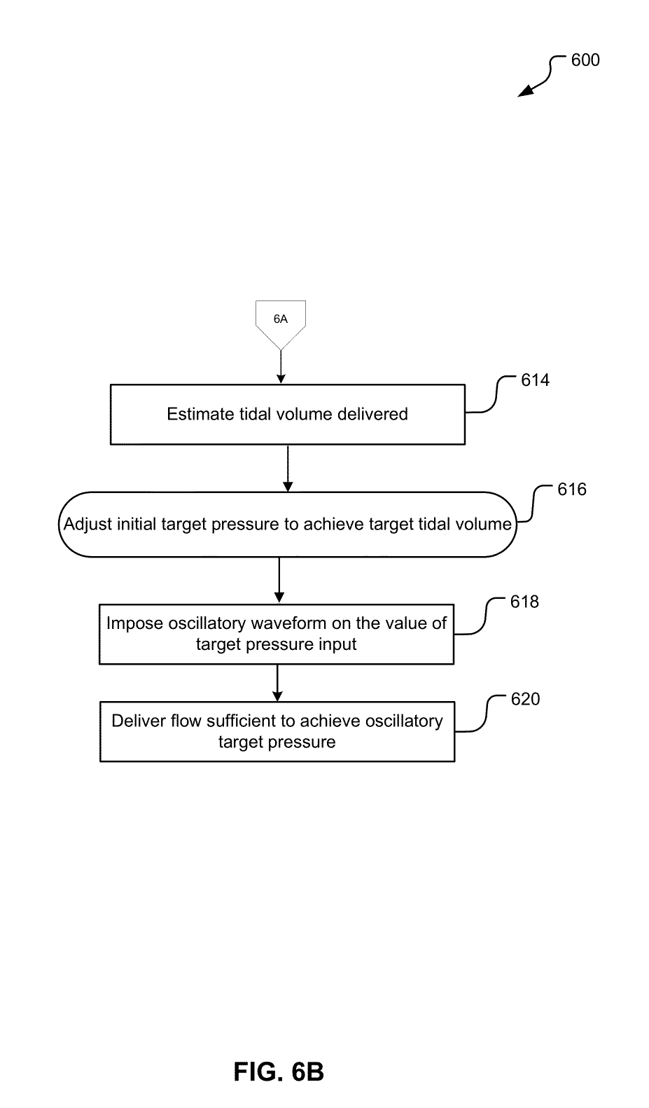

FIGS. 6A-6B are flow charts illustrating a further embodiment of a method for providing oscillatory pressure controlled delivery of gas flow to a patient during pressure regulated volume targeted ventilation. In further embodiments, pressure regulated volume targeted ventilation with imposed waveform oscillations is provided. After delivery of a breath, a volume estimation may be determined as to how much volume is delivered into the patient's lungs. For a next breath, based on the determined estimated volume information (and/or any other parameters determined during a first breath), first oscillatory target pressure may be increased or decreased to increase or decrease the delivered pressure. Breath to breath, target pressure may be adjusted to reach a target volume. For example, ventilation module 212 may increase the first target pressure or decrease the first target pressure so that the volume that enters the patient's lungs is a set value. If oscillatory target pressure is increased, more volume may be delivered. If oscillatory target pressure is decreased, less volume may be delivered. Oscillatory pressure control module 224 may adjust the first oscillatory target pressure delivered adaptively so that after several breaths, the target has reached a level such that the volume that is delivered to the patient is the set volume.

Method 600 begins with ventilation setup operation 602. According to embodiments, ventilation involves delivering breathing gases to a patient who is unable to breathe completely independently. Ventilation includes delivering any portion of breathing gases, from full mandatory ventilation to full or partially-supported spontaneous ventilation.

At receive operation 604, a target tidal volume input may be received. During setup, an operator may select a volume of gas to be delivered to the patient, (e.g., delivered into the lung), over a specified time period such as a minute, a number of breaths, etc. In some instances, a specific respiratory volume value may be input via user interface 210. This volume value is received by the ventilator and may be stored in memory 208 for use during ventilation.

At receive operation 606, a first target pressure input may be received. As described above, ventilation module 212 may determine a target pressure level that will deliver the desired volume into the lungs. During a breathing phase, the target pressure may be set to an initial target pressure value via inhalation module 214. For instance, inhalation module 214 may receive an inhalation pressure desired value. This inhalation pressure desired value may be stored in memory 208. The desired value for the exhalation pressure may be received by exhalation module 216 and may also be stored in memory 208. Target pressure may be an oscillatory target pressure, as discussed above.

At receive operation 608, an oscillation parameter input may be received. Oscillatory target pressure may be configured with one or more oscillation parameters as described above. To this end, input breathing phase time, tidal volume amount, target pressure and any other parameters may be received by, for example, oscillatory pressure control module 224. Other parameters, including total breath time, rise time, oxygen concentration, and breath rate may be determined at setup. Any of the ventilation parameters (e.g., target pressure, maximum pressure, breath duration, etc.) may be constant (e.g., subject to a set desired volume) or may adaptively adjust to comply with one or more parameters (e.g., satisfy delivery of a certain volume while oscillating about the target). One or more input parameters may also adapt based on changes in patient behavior (e.g., spontaneous breathing). Delivered volume may remain constant or substantially constant while the target pressure per breathing phase for each breath oscillates according to a previously determined setting.

At impose operation 610, an oscillatory waveform may be imposed on the target pressure. Oscillatory pressure control module 224 may then generate and impose an oscillating waveform. The oscillating waveform may include upper and lower bounds defined by the oscillation parameters. Individual oscillations of the oscillatory waveform may also be further defined by one or more oscillation parameters as described above. To this end the oscillating waveform may be configured to oscillate substantially about the first target pressure for a set duration, including an inhalation, a plurality of consecutive inhalations, a single exhalation, a plurality of consecutive exhalations, a single breath or a plurality of consecutive breaths, or any other desired pattern of inhalation and/or exhalation. In some embodiments, oscillatory pressure control module 224 may impose the oscillating waveform on the value of the first target pressure for at least a portion of the received or detected time duration for the single inhalation or exhalation. For instance, the first target pressure value may be utilized in any suitable algorithm configured to generate an oscillatory target pressure based on the one or more input oscillation parameters. For instance, in embodiments where the waveform is sinusoidal, and oscillatory pressure is provided for an inspiration, target pressure may be defined by the following equation:

Target Pressure=Pₘₑₐₙ+Pₐₘₚₗ*sin(2pi*tᵢ*F)

Where Pₘₑₐₙ=target pressure, Pₐₘₚₗ=Pₘₐₓ−Pₘₑₐₙ, pi=3.1415926, tᵢ=inspiratory time, F=sinusoid frequency=1/T, and T=sinusoid period.

Determined oscillatory target pressure values may then be utilized by oscillatory pressure control module 224 to modulate the first target pressure according to the algorithm. In some embodiments, oscillatory pressure control module 224 may be configured to increase the input target pressure up to the maximum pressure input value for half the waveform cycle duration and decrease inspiratory pressure an amount substantially equivalent to the pressure increase for the remaining time of the inspiratory waveform duration. Thus, the delivered pressure may oscillate (e.g., incrementally increase or decrease) substantially about the input target pressure during a single inhalation, a single exhalation, or a single breath according to the one or more pre-determined or adaptive oscillation parameters described above. When the input time duration has expired, or when an inspiration or exhalation end is detected, oscillatory pressure control module 224 may cease delivery of oscillatory pressure until a next time period for delivery is detected.

At deliver operation 612, a first amount of flow sufficient to achieve the first target pressure may be delivered. To provide pressure regulated volume targeted ventilation with oscillatory pressure control, an initial amount of pressure may be delivered to the patient during an inspiration or exhalation phase (via, for example, ventilation module 212).

At estimate operation 614, an amount of tidal volume delivered is estimated. After delivery of a breath, a delivered tidal volume may be estimated. A delivered volume may be measured, for instance, by one or more distributed sensors 218, or one or more internal sensors 220. The estimated tidal volume may be compared to the target volume value to determine if the delivered volume is less than or greater than the target volume.

At adjust operation 616, the first target pressure input may be adjusted to achieve the target tidal volume. The first oscillatory target pressure may be increased or decreased by a preselected pressure increment or another algorithm such as any appropriate form of closed-loop dynamic regulation during the next breathing phase. The first oscillatory target pressure value may be increased or decreased until the delivered volume is substantially equal to the volume target (e.g., to maintain a constant volume of delivered gas that enters the patient's lungs). Increasing the first oscillatory target pressure may provide an indication that more volume is to be delivered. Decreasing the first oscillatory target pressure may provide an indication that less volume is to be delivered. The delivered oscillatory pressure may also be adjusted adaptively so that after several breaths, the volume delivered to the patient has reached a level equivalent or substantially equivalent to the set volume. Oscillations may similarly adjust (e.g., increase or decrease in amplitude and/or frequency) according to the target pressure for each breath based on the set volume target desired across multiple breaths.

At impose operation 618, an oscillatory waveform may be imposed on the adjusted target pressure. Oscillatory pressure control module 224 may generate and impose an oscillating waveform onto the adjusted target pressure. The oscillating waveform may include upper and lower bounds defined by the original oscillation parameters, may be modified by an operator, or may adaptively adjust based on changes in patient behavior. Individual oscillations of the oscillatory waveform may also be further defined by one or more oscillation parameters as described above. To this end the oscillating waveform may be configured to oscillate substantially about the adjusted target pressure for a set duration, including an inhalation, a plurality of consecutive inhalations, a single exhalation, a plurality of consecutive exhalations, a single breath or a plurality of consecutive breaths, or any other desired pattern of inhalation and/or exhalation. In some embodiments, oscillatory pressure control module 224 may impose the oscillating waveform on the value of the adjusted target pressure for at least a portion of the received or detected time duration for the single inhalation or exhalation. For instance, the adjusted target pressure value may be utilized in any suitable algorithm configured to generate an oscillatory target pressure based on the one or more input oscillation parameters. For instance, the waveform may be sinusoidal and may be defined as described above.

At deliver operation 620, a second amount of flow sufficient to achieve the adjusted target pressure may be delivered. Second amount of flow may be greater or less than initial flow amount delivered, based on the adjusted target pressure.

Determined oscillatory target pressure values may then be utilized by oscillatory pressure control module 224 to modulate the adjusted target pressure according to the algorithm. In some embodiments, oscillatory pressure control module 224 may be configured to increase the adjusted target pressure up to the maximum pressure input value for half the waveform cycle duration and decrease inspiratory pressure an amount substantially equivalent to the pressure increase for the remaining time of the inspiratory waveform duration. Thus, the delivered pressure may oscillate (e.g., incrementally increase or decrease) substantially about the adjusted target pressure during a single inhalation, a single exhalation, or a single breath according to the one or more pre-determined or adaptive oscillation parameters described above. When the input time duration has expired, or when an inspiration or exhalation end is detected, oscillatory pressure control module 224 may cease delivery of oscillatory pressure until a next time period for delivery is detected.

As should be appreciated, the particular steps and methods described above with reference to FIGS. 6A-6B are not exclusive and, as will be understood by those skilled in the art, the particular ordering of steps as described herein is not intended to limit the method, e.g., steps may be performed in differing order, additional steps may be performed, and disclosed steps may be excluded without departing from the spirit of the present methods.

Those skilled in the art will recognize that the methods and systems of the present disclosure may be implemented in many manners and as such are not to be limited by the foregoing exemplary embodiments and examples. In other words, functional elements being performed by a single or multiple components, in various combinations of hardware and software or firmware, and individual functions, can be distributed among software applications at either the client or server level or both. In this regard, any number of the features of the different embodiments described herein may be combined into single or multiple embodiments, and alternate embodiments having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter.

Numerous other changes may be made which will readily suggest themselves to those skilled in the art and which are encompassed in the spirit of the disclosure and as defined in the appended claims. While various embodiments have been described for purposes of this disclosure, various changes and modifications may be made which are well within the scope of the present invention. Numerous other changes may be made which will readily suggest themselves to those skilled in the art and which are encompassed in the spirit of the disclosure and as defined in the claims.

We claim:

1. A method for ventilating a patient with a ventilator comprising:
receiving a target pressure input for a breathing phase;
receiving at least one oscillation parameter;
imposing an oscillatory waveform on the target pressure, the oscillatory waveform having characteristics defined by the at least one oscillation parameter and configured to oscillate substantially about the target pressure for at least a portion of the breathing phase; and
delivering an amount of flow sufficient to achieve an oscillatory target pressure based on the imposed oscillatory waveform.

2. The method of claim 1, wherein the receiving at least one oscillation parameter includes:
receiving a maximum pressure input.

3. The method of claim 1, wherein the receiving at least one oscillation parameter includes:
receiving an oscillatory waveform shape selection.

4. The method of claim 1, wherein the receiving at least one oscillation parameter includes:
receiving an oscillation cycle duration parameter.

5. The method of claim 1, wherein imposing an oscillatory waveform on the target pressure includes:
imposing the at least one oscillation parameter during at least one of inhalation or exhalation.

6. The method of claim 1, wherein imposing an oscillatory waveform on the target pressure includes:
imposing the at least one oscillation parameter for a fixed duration.

7. The method of claim 1, further including:
stochastically determining the at least one oscillation parameter based on a patient behavior.

8. The method of claim 1, further including:
adaptively adjusting the at least one oscillation parameter from inhalation to exhalation.

9. A ventilator system comprising:
a pressure generating system adapted to generate a flow of breathing gas;
a ventilation tubing system including a patient interface for connecting the pressure generating system to a patient;
at least one sensor operatively coupled to at least one of the pressure generating system, the patient, and the ventilation tubing system;
an oscillatory pressure control module configured to receive a target pressure input for a breathing phase, receive at least one oscillation parameter, detect that an amount of flow sufficient to achieve the target pressure has been achieved, impose an oscillatory waveform on the target pressure, the oscillatory waveform having characteristics defined by the at least one oscillation parameter and configured to oscillate substantially about the target pressure for at least a portion of a breathing phase, and deliver an amount of flow sufficient to achieve an oscillatory target pressure based on the imposed oscillatory waveform; and
a processor in communication with the pressure generating system, the at least one sensor, and the oscillatory pressure control module.

10. The ventilator system of claim 9, wherein the one or more oscillation parameters defining the oscillatory waveform include at least one of a mean target pressure input, a maximum target pressure input, a total duration input and a total oscillation duration value.

11. The ventilator system of claim 9, wherein the one or more oscillation parameters defining the oscillatory waveform include an oscillatory waveform shape.

12. The ventilator system of claim 9, wherein oscillatory pressure control module imposes the one or more oscillation parameters defining the oscillatory waveform during at least one of inhalation or exhalation.

13. The ventilator system of claim 9, wherein the one or more oscillation parameters defining the oscillatory waveform are fixed.

14. The ventilator system of claim 9, wherein the parameters of the one or more oscillation parameters defining the oscillatory waveform are stochastically determined.

15. The ventilator system of claim 9, wherein the one or more oscillation parameters defining the oscillatory waveform vary from inhalation to exhalation.

16. The ventilator system of claim 9, wherein the oscillatory waveform is at least one of a sinusoidal waveform, a square waveform, a rectangular waveform, a triangular waveform, a sawtooth waveform, a pulse-type waveform or a trigger-type waveform.

17. A computer-readable medium having computer-executable instructions for performing a method for ventilating a patient with a ventilator, the method comprising:
receiving a target pressure input for a breathing phase;
receiving at least one oscillation parameter;
imposing an oscillatory waveform on the target pressure, the oscillatory waveform having characteristics defined by the at least one oscillation parameter and configured to oscillate substantially about the target pressure for at least a portion of the breathing phase; and
delivering an amount of flow sufficient to achieve an oscillatory target pressure based on the imposed oscillatory waveform.

18. The computer-readable medium of claim 17, wherein the receiving at least one oscillation parameter includes at least one of:
receiving a maximum pressure input;
receiving an oscillatory waveform shape selection; and
receiving an oscillation cycle duration parameter.

19. The computer-readable medium of claim 17, wherein imposing an oscillatory waveform on the target pressure includes:
imposing the at least one oscillation parameter during at least one of inhalation or exhalation.

20. A method for ventilating a patient with a ventilator comprising:
receiving a target tidal volume input;
receiving a first target pressure input;
receiving at least one oscillation parameter;
imposing a first oscillatory waveform on the target pressure, the first oscillatory waveform defined by the at least one oscillation parameter and configured to oscillate substantially about the first target pressure for at least a portion of a first breathing phase;
delivering a first amount of flow sufficient to achieve a first oscillatory target pressure based on the first imposed oscillatory waveform;
after a first flow delivery is substantially complete, estimating an amount of volume delivered;
based on the estimated delivered volume, adjusting the first pressure input;
imposing a second oscillatory waveform on the adjusted target pressure, the second oscillatory waveform configured to oscillate substantially about the adjusted target pressure for at least a portion of a second breathing phase; and
delivering a second amount of flow sufficient to achieve an adjusted target pressure based on the second imposed oscillatory waveform.
